(19) United States
(12) Patent Application Publication
(10) Pub. No.: US 2026/0305619 A1
(43) Pub. Date: Oct. 8, 2026

(54) METHODS OF WEED CONTROL

(71) Applicant: Weedout Ltd., Nes Ziona (IL)

(72) Inventors: Efrat LIDOR-NILI, Nes Ziona (IL); Orly NOIVIRT-BRIK, Givataim (IL); Ido SHWARTZ, Herzeliya (IL); Amit KOCH, Rehovot (IL); Miriam AMINIA, Kibbutz Nirim (IL); Tarryn SCHULDINER-HARPAZ, Rehovot (IL)

(73) Assignee: Weedout Ltd., Nes Ziona (IL)

(21) Appl. No.: 19/476,191

(22) PCT Filed: Apr. 17, 2024
(86) PCT No.: PCT/IL2024/050381

§ 371 Date: Oct. 17, 2025

Related U.S. Application Data
(60) Provisional application No. 63/459,717, filed on Apr. 17, 2023.

(51) Int. Cl. A01N 65/08; A01P 13/00

(52) U.S. Cl. CPC A01N 65/08; A01P 13/00

(57) ABSTRACT

According to an aspect of some embodiments of the present invention there is provided a method of weed control. The method comprising artificially pollinating a weed species of interest with irradiated pollen that reduces fitness of the weed species of interest, the pollen being of a different species than that of the weed species of interest and capable of competing with pollen of the weed species of interest, wherein the artificially pollinating is effected under competition conditions with native pollen.

### RELATED APPLICATIONS

This application claims priority from U.S. Provisional Patent Application No. 63/459,717 filed on Apr. 17, 2023, which is hereby incorporated by reference in its entirety.

### FIELD AND BACKGROUND OF THE INVENTION

The present invention, in some embodiments thereof, relates to methods of weed control and specifically the use of xeno pollen in weed control.

Weeds have been the major biotic cause of crop yield loses since the origins of agriculture. The potential of weed damages is estimated as 34% loss of crop yield, on average, world-wide [Oerke, E-C., 2006]. In the USA alone, the annual cost of crop losses due to weeds is greater than 26 billion USD [Pimentel D et al., 2000]. Furthermore according to the Weed Science Society of America Weeds are estimated to cause more than 40 billion USD in annual global losses [wssa (dot) net/wssa/weed/biological-control/]. Weeds are thus a major threat to food security [Delye et al., 2013].

Herbicides are the most commonly used and effective weed control tools. Due to the intense selection pressure exerted by herbicides, herbicide resistance is constantly growing and as of 2016 there are over 470 weed biotypes currently identified as being herbicide resistant to one or more herbicides by The International Survey of Herbicide Resistant Weeds (weedscience (dot) org/).

Weeds, like other plants, have several sexual reproduction mechanisms: self-pollination, cross-pollination, or both. Self-pollination describes pollination using pollen from one flower that is transferred to the same or another flower of the same plant. Cross-pollination describes pollination using pollen delivered from a flower of a different plant. Weeds rely on wind, or animals such as bees and other insects to pollinate them.

Since the 1940's the use of sterile organisms has been reported for use in order to reduce pest population and the success of these methods was demonstrated in many cases such as the tsetse fly [Klassen& Curtis, 2005], melon fly [Yosiakiet al. 2003] and Sweet potato weevil [Kohama et al., 2003].

Planting in the field plants producing sterile pollen for the production of infertile seeds was mentioned but immediately over-ruled due to practical, regulatory and economic reasons.

PCT Publication No. WO2017/203519 discloses a method of weed control comprising artificially pollinating a weed species of interest with pollen of the same species that reduces fitness of the weed species of interest, thereby blocking the next generation of viable weed seeds. PCT Publication No. WO2020/084586 discloses a method of inhibiting growth of weeds.

### Additional Background Art Includes:

Gaines, T. A et al. (2012) Interspecific hybridization transfers a previously unknown glyphosate resistance mechanism in Amaranthus species. Evolutionary Applications 5 (1): 29-38. Trucco et al. 2007 Weed Science 55:119-122.

### SUMMARY OF THE INVENTION

According to an aspect of some embodiments of the present invention there is provided a method of weed control, the method comprising artificially pollinating a weed species of interest with irradiated pollen that reduces fitness of the weed species of interest, the pollen being of a different species than that of the weed species of interest and capable of competing with pollen of the weed species of interest, wherein the artificially pollinating is effected under competition conditions with native pollen.

According to some embodiments of the invention, the pollen consists of pollen of a different species than that of the weed species of interest.

According to some embodiments of the invention, the artificially pollinating is effected in a large scale of at least 0.1 acre.

According to some embodiments of the invention, the pollen is comprised in a formulation which allows pollen distribution by artificial pollination.

According to some embodiments of the invention, the irradiated pollen is by y radiation.

According to some embodiments of the invention, the irradiated pollen is by X radiation.

According to some embodiments of the invention, the irradiated pollen is by alpha radiation.

According to some embodiments of the invention, the pollen is of the same genus as the weed species of interest.

According to some embodiments of the invention, the pollen and the weed species of interest are of different genera.

According to some embodiments of the invention, at least one of said pollen and said weed species of interest is a hybrid.

According to some embodiments of the invention, the pollen is non-genetically modified pollen.

According to some embodiments of the invention, the pollen is genetically modified pollen.

According to some embodiments of the invention, the pollen is of the Amaranthaceae family.

According to some embodiments of the invention, the pollen is of the Amaranthus genus.

According to some embodiments of the invention, the pollen is A. palmeri.

According to some embodiments of the invention, the pollen and the weed species of interest are selected from the group consisting of A. palmeri and A. tuberculatus.

According to some embodiments of the invention, the pollen is A. palmeri.

According to some embodiments of the invention, the pollen is a hybrid of A. palmeri and A. tuberculatus.

According to some embodiments of the invention, the weed species of interest is a hybrid of A. palmeri and A. tuberculatus.

According to some embodiments of the invention, the pollen is of the Lolium genus.

According to some embodiments of the invention, the pollen is of the Alopecurus genus.

According to some embodiments of the invention, the pollen and the weed species of interest are selected from the group of pairs shown in Table 1.

According to some embodiments of the invention, the capable of competing is by fertilizing the weed species of interest.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

### DESCRIPTION OF SPECIFIC EMBODIMENTS OF THE INVENTION

The present invention, in some embodiments thereof, relates to methods of weed control.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have previously devised a technology for weed control using irradiated pollen of the same species as the target weed. Such pollen is typically applied during weed flowering period via artificial pollination to prevent the generation of viable resistant weed seeds, while competing with wild pollen which is present in the field.

Whilst further developing the technology, the present inventors have surprisingly found that pollen of weed of a different species than the target weed can be effectively used in a method of weed control. This is surprising since it is not at all expected that an irradiated pollen outside the species of the target plant can effectively fertilize and subsequently elicit a similar seed set of hybrid weed in a manner comparable to that when irradiated pollen and even non-irradiated pollen (see Examples section) of the same species is applied. In both cases the resultant seeds are aborted and can't effectively germinate (see e.g., Example 1). These results were reinforced in experiments which were done under competition conditions, showing the ability of the irradiated pollen to compete successfully with non-irradiated pollen of the target plant (same species, see e.g., Example 2). As such, pollen of the present invention which is outside (i.e., xeno) the species of the target weed can be used as a universal pollen for weed control.

Thus, according to an aspect of the invention there is provided a method of weed control. The method comprising artificially pollinating a weed species of interest with irradiated pollen that reduces fitness of the weed species of interest, the pollen being of a different species than that of the weed species of interest and capable of competing with pollen of the weed species of interest, wherein the artificially pollinating is effected under competition conditions with native pollen.

As used herein “competing” refers to the ability of the pollen to reduce the viable seed set resultant from natural fertilization by the pollen of the target. This can be done by myriads of ways such as physically occupying the position of the target pollen on the female organ and/or fertilizing the target plant. Thus, the seed set can be about the same in terms of amount but the number of non-viable seed in the seed set (e.g., as manifested by seed weight and/or ability to germinate) is higher than that of the control.

As used herein “control” refers to fertilization by a non-irradiated pollen of the same species as that of the weed species of interest (also referred to as “target” or “target plant”).

As used herein the phrase “weed control” refers to suppressing growth and optionally spread of a population of at least one weed species of interest or a member or members of a genus or a family thereof and even reducing the size of their population in a given growth area.

According to some embodiments of the invention, the weed control is effected at a growth area of at least 0.1 acre and optionally not exceeding 100,000 acres, e.g., 50,000 acres.

According to a specific embodiment, the growth is 1-10000 acres, e.g., 1-5000, 1-2000, 1-1000 acres.

According to some embodiments of the invention, the weed control is effected at a growth area of at least 1 acre.

According to a specific embodiment, the growth area is an urban area, e.g., golf courses, athletic fields, parks, cemeteries, roadsides, home gardens/lawns and the like.

According to an additional or alternative embodiment, the growth area is a rural area.

According to an additional or an alternative embodiment, the growth area is an agricultural growth area e.g., open field, greenhouse, plantation, vineyard, orchard and the like.

As used herein the term “weed species of interest” refers to a wild plant growing where it is not wanted and that may be in competition with cultivated plants of interest (i.e., crop-desirable plants). Weeds are typically characterized by rapid growth and/or ease of germination, and/or competition with crops for space, light, water and nutrients.

According to some embodiments of the invention, the weed species of interest is traditionally non-cultivated. The weed species of interest may also be referred to as the target weed.

According to another embodiment of the invention, the weed of interest or irradiated pollen is a perennial weed.

According to another embodiment of the invention the weed of interest or irradiated pollen is a biennial weed.

According to another embodiment of the invention the weed of interest or irradiated pollen is an annual weed.

According to another embodiment of the invention the weed of interest or irradiated pollen is a therophyte.

According to an embodiment, the weed of interest or irradiated pollen is a parasitic plant.

According to an embodiment, the weed of interest or irradiated pollen reproduces sexually.

According to an embodiment, the weed of interest or irradiated pollen is a broadleaf weed.

According to an embodiment, the weed of interest or irradiated pollen is a grassy weed.

According to an embodiment, the weed of interest or irradiated pollen is a non-grass monocot weed.

According to an embodiment, the weed of interest or irradiated pollen is of a monoecious plant.

According to an embodiment, the weed of interest or irradiated pollen is of a dioecious plant.

According to an embodiment, the weed of interest or irradiated pollen is of a protogynous plant, in which the flowering of the male flowers and female flowers is spatially or temporally regulated. For example, female flowers that mature before male flowers, ensuring a high level of outcrossing (temporal regulation) are found in Bassia scoparia (kochia). The genus Amaranthus L. is a diverse plant group of 70-80 species distributed across the world's temperate and tropical regions [Raiyemo et al. BMC Biology (2023) 21:37]. Nine of these species [Amaranthus acanthochiton J. D. Sauer, Amaranthus Arenicola I. M. Johnson, Amaranthus australis (A. Gray) J. D. Sauer, Amaranthus cannabinus (L.) J. D. Sauer, Amaranthus floridanus (S. Watson) J. D. Sauer, Amaranthus tuberculatus (Moq.) J. D. Sauer, Amaranthus greggii S. Watson, Amaranthus watsonii Standley, and Amaranthus palmeri S. Watson] are dioecious (i.e., separate male and female individual plants), native to North America and grouped collectively into the subgenus Acnida (L.) Aellen ex K. R. In monoecious, which are also contemplated herein, both male and female flowers can be found on one plant.

As used herein “genus” refers to a plurality of species.

As used herein “family” refers to a plurality of genera. Family is a basic taxonomic grouping ranking above genus and below order.

Typically, the classification is a Linnaeus classification system.

Examples of weed families include, but are not limited to, Amaranthaceae, Anacardiaceae, Compositae, Apocynaceae, Curcurbitaceae, Poaceae (Graminaceae), Cannabaceae, Chenopodiaceae, Leguminosae; Fabaceae, Lythraceae, Malvaceae, Asclepiadaceae, Labiatae, Convolvulaceae, Cruciferae, Solanaceae, Plantaginaceae, Portulacaceae, Rosaceae, Cyperaceae and Polygoneaceae.

Examples of weed species which can be targeted (mitigated) according to the present teachings include, but are not limited to, Amaranthus species-A. albus, A. blitoides, A. hybridus, A. palmeri, A. powellii, A. retroflexus, A. rudis, A. spinosus, A. tuberculatus, A. thunbergii, A. graecizans and A. viridis; Ambrosia species-A. trifida, A. artemisifolia; Lolium species-L. multiflorum, L. rigidium, L perenne; Digitaria species-D. insularis, D. sanguinalis; Euphorbia species-E. heterophylla; Kochia species-K. scoparia;Sorghum species-S. halepense; Conyza species-C. bonariensis, C. canadensis, C. sumatrensis; Chloris species-C. truncate; Echinochola species-E. colona, E. crus-galli; Eleusine species-E. indica; Poa species-P. annua; Plantago species-P. lanceolata; Avena species-A. fatua;Chenopodium species-C. album; Setaria species-S. viridis, Abutilon theophrasti, Ipomoea species, Sesbania, species, Xanthium strumarium, Cassia species, Sida species, Brachiaria species, Sporobolus species-S. pyramidalis, S. natalensis, S. jacquemontii, S. fertilis, S. africanus S. indicus, Solanum nigrum, Solanum carolinense, and Solanum elaeagnifolium.

Additional weedy plant species found in cultivated areas include Alopecurus myosuroides, Avena sterilis, Avena sterilis ludoviciana, Brachiaria plantaginea, Bromus diandrus, Bromus rigidus, Cynosurus echinatus, Digitaria ciliaris, Digitaria ischaemum, Digitaria sanguinalis, Echinochloa oryzicola, Echinochloa phyllopogon, Eriochloa punctata, Hordeum glaucum, Hordeum leporinum, Ischaemum rugosum, Leptochloa chinensis, Lolium persicum, Phalaris minor, Phalaris paradoxa, Rottboellia exalta, Setaria faberi, Setaria viridisvar, robusta-alba schreiber, Setaria viridisvar, robusta-purpurea, Snowdenia polystachea, Sorghum Sudanese, Alisma plantago-aquatica, Amaranthus lividus, Ammaniaauriculata, Ammania coccinea, Anthemis cotula, Apera spica-venti, Bacopa rotundifolia, Bidens pilosa, Bidens subalternans, Brassica tournefortii, Bromus tectorum, Camelina microcarpa, Chrysanthemum coronarium, Cuscuta campestris, Cyperus dijformis, Damasonium minus, Descurainia sophia, Diplotaxis tenuifolia, Echium plantagineum, Elatine triandravar, pedicellata, Euphorbia heterophylla, Fallopia convolvulus, Fimbristylis miliacea, Galeopsis tetrahit, Galium spurium, Helianthus annuus, Iva xanthifolia, Ixophorusunisetus, Ipomoea indica, Ipomoea purpurea, Ipomoea sepiaria, Ipomoea aquatic, Ipomoea triloba, Lactuca serriola, Limnocharis flava, Limnophila erecta, Limnophila sessiliflora, Lindernia dubia, Lindernia dubiavar, major, Lindernia micrantha, Lindernia procumbens, Mesembryanthemum crystallinum, Monochoria korsakowii, Monochoria vaginalis, Neslia paniculata, Papaver rhoeas, Parthenium hysterophorus, Pentzia suj fruticosa, Phalaris minor, Raphanus raphanistrum, Raphanus sativus, Rapistrum rugosum, Rotalaindicavar, uliginosa, Sagittaria guyanensis, Sagittaria montevidensis, Sagittaria pygmaea, Salsola iberica, Scirpus juncoidesvar, ohwianus, Scirpus mucronatus, Setaria lutescens, Sida spinosa, Sinapis arvensis, Sisymbrium orientale, Sisymbrium thellungii, Solanum ptycanthum, Sonchus asper, Sonchus oleraceus, Sorghum bicolor, Stellaria media, Thlaspi arvense, Xanthium strumarium, Arctotheca calendula, Conyza sumatrensis, Crassocephalum crepidiodes, Cuphea carthagenenis, Epilobium adenocaulon, Erigeron philadelphicus, Landoltia punctata, Lepidium virginicum, Monochoria korsakowii, Solanum americanum, Solanum nigrum, Vulpia bromoides, Youngia japonica, Hydrilla verticillata, Carduus nutans, Carduus pycnocephalus, Centaurea solstitialis, Cirsium arvense, Commelina diffusa, Convolvulus arvensis, Daucuscarota, Digitaria ischaemum, Echinochloa crus-pavonis, Fimbristylis miliacea, Galeopsis tetrahit, Galium spurium, Limnophila erecta, Matricaria perforate, Papaver rhoeas, Ranunculus acris, Soliva sessilis, Sphenoclea zeylanica, Stellaria media, Nassella trichotoma, Stipa neesiana, Agrostis stolonifera, Polygonum aviculare, Alopecurus japonicus, Beckmannia syzigachne, Bromus tectorum, Chloris inflate, Echinochloa erecta, Portulaca oleracea, and Senecio vulgaris. Examples of parasitic plants include, but are not limited to, Striga sp, Orobanche sp, Cuscuta sp, Mistletoe.

According to a specific embodiment the weed species is selected from or belong to the group consisting of Amaranthus: A. palmeri, A. tuberculatus, A. retroflexus, A. hybridus, A. powellii, A. spinosus, A. albus, A. blitoides, A. rudis, Lolium rigidum, Lolium multiflorum, Lolium perenne Ambrosia: A. trifida, A. artemisiifolia, Kochia scoparia, Conyza: C. canadensis, C. bonariensis, Echinochloa, Alopecurus myosuroides, Sorghum halepense, Digitaria insularis, Eleusine indica, Avena fatua, Euphorbia Heterophylla and Chenopodium album.

According to a specific embodiment, the pollen is of the Amaranthaceae family.

According to a specific embodiment, the pollen is of the Amaranthus genus.

According to a specific embodiment, the pollen is of the Poaceae family.

According to a specific embodiment, the pollen is of the Lolium genus.

According to a specific embodiment, the pollen is of the Alopecurus genus.

According to a specific embodiment, the pollen is of the Ambrosia genus.

According to a specific embodiment, the pollen is of the Erigeron genus.

According to a specific embodiment, the pollen is of the Asteraceae family.

According to a specific embodiment, at least one of said pollen and said weed species of interest is hybrid. Thus, the pollen can be hybrid pollen, the weed species of interest can be a hybrid or both can be hybrids, albeit different hybrids.

According to a specific embodiment, said pollen is A. palmeri.

According to a specific embodiment, said pollen is a hybrid of A. palmeri and A. tuberculatus.

According to a specific embodiment, said weed species of interest is a hybrid of A. palmeri and A. tuberculatus.

According to a specific embodiment, said pollen and said weed species of interest are selected from the group consisting of A. palmeri and A. tuberculatus. Thus the pollen may be A. palmeri or a hybrid thereof and the weed species of interest A. tuberculatus or a hybrid thereof. The weed species of interest may be A. palmeri or a hybrid thereof and the pollen A. tuberculatus or a hybrid thereof.

Other examples of Amaranth hybrids include, but are not limited to A. palmeri x A. spinosus, A. palmeri x A. hybridus, A. tuberculatus x A. hybridus, A. tuberculatus x A. powellii, A. tuberculatus x retroflexus, and A. tuberculatus x A. spinosus.

According to a specific embodiment, the pollen or the target is not hybrid or specifically, any one of the hybrids mentioned herein.

As mentioned the pollen is not of the same species as the target weed, yet within the family as the target weed. The pollen can be of a different species of the genus as the target weed. Alternatively, the pollen can be of a different genus than that of the target weed, yet, under embodiments of the invention, the pollen is of the same family as the target weed. For example, C. album with A. palmeri.

According to a specific embodiment, the pollen is a mixture of pollen (e.g., 2, 3, 4, 5) of different species or genera than the target weeds in the treated field. For instance more than 40%, 50%, 60%, 70% of the weed in the treated area are not of the same species as the pollen used in weed control.

According to a specific embodiment, the pollen consists of pollen of a different species than that of the weed species of interest (as mentioned, the target weed).

Different weed may have different growth habits and therefore specific weeds usually characterize a certain crop in given growth conditions.

According to a specific embodiment, the weed is a herbicide resistant weed.

According to a specific embodiment, weed is defined as herbicide resistant when it meets the Weed Science Society of America (WSSA) definition of resistance.

Accordingly, WSSA defines herbicide resistance as “the inherited ability of a plant to survive and reproduce following exposure to a dose of herbicide normally lethal to the wild type. Alternatively, herbicide resistance is defined as “The evolved capacity of a previously herbicide-susceptible weed population to withstand an herbicide and complete its life cycle when the herbicide is used at its normal rate in an agricultural situation” (Source: Heap and Lebaron. 2001 in Herbicide Resistance and World Grains).

As mentioned, weed control according to the present teachings is effected by reducing fitness of the at least one weed species of interest.

As used herein “fitness” refers to the relative ability of the weed species of interest to develop, reproduce or propagate and transmit its genes to the next generation. As used herein “relative” means in comparison to a weed of the same species not having been artificially pollinated with the pollen of the invention and grown under the same conditions.

It will be appreciated that the effect of pollen treatment according to the present teachings is typically manifested in the first generation after fertilization. As shown in the Examples section, fertilizing with irradiated xeno pollen of the present invention, elicited an aborted seed set manifested by significantly lower weight (results which were further corroborated under competition) and poorer ability to germinate as compared to control seed set. As mentioned above, the control is non-irradiated pollen of the same species as that of the target weed, such a control reflects natural competition conditions. According to other embodiments, the control is irradiated pollen of the same species as that of the target weed.

The fitness may be affected by reduction in productiveness, propagation, fertility, fecundity, biomass, biotic stress tolerance, abiotic stress tolerance and/or herbicide resistance.

As used herein “productivity” refers to the potential rate of incorporation or generation of energy or organic matter by an individual, population or trophic unit per unit time per unit area or volume; rate of carbon fixation.

As used herein “fecundity” refers to the potential reproductive capacity of an organism or population, measured by the number of gametes.

According to a specific embodiment, the pollen affects any stage of seed development or germination.

According to a specific embodiment, the reduction in productiveness is manifested by at least one of:
(i) inability to develop an embryo;
(ii) embryo abortion;
(iii) seed non-viability;
(iv) seed that cannot fully develop; and/or
(v) seed that is unable to germinate.

According to a specific embodiment, the reduction in productiveness is manifested by at least one of:
(i) inability to develop an embryo;
(ii) embryo abortion;
(iii) seed non-viability;
(iv) seed that cannot fully develop;
(v) seed that is unable to germinate; and/or
(vi) reduced or no seed set.

It will be appreciated that when pollen reduces the productiveness, fertility, propagation ability or fecundity of the weed in the next generation it may be referred to by the skilled artisan as sterile pollen, though it fertilizes the weed of interest. Hence, sterile pollen as used herein is still able to fertilize but typically leads to seed developmental arrest or seed abortion.

According to a specific embodiment, the seed set is about similar in quantity to that obtained when using irradiated pollen or non-irradiated of the same species as the target weed. +/−0.1-5 fold (e.g., 0.4-5 fold).

According to a specific embodiment, the reduction in fitness is by at least 10%, 20%, 30%, 40%, 50%, 60%, 70%, 75%, 80%, 85%, 90%, 92%, 95%, 97% or even 100%, within first generation after fertilization and optionally second generation after fertilization and optionally third generation after fertilization.

According to a specific embodiment, the reduction in fitness is by at least 10%, 20%, 30%, 40%, 50%, 60%, 70%, 75%, 80%, 85%, 90%, 92%, 95%, 97% or even 100%, within first generation after fertilization.

According to a specific embodiment, reduced fitness results from reduction in tolerance to biotic or abiotic conditions e.g., herbicide resistance.

Non-limiting examples of abiotic stress conditions include, salinity, osmotic stress, drought, water deprivation, excess of water (e.g., flood, waterlogging), etiolation, low temperature (e.g., cold stress), high temperature, heavy metal toxicity, anaerobiosis, nutrient deficiency (e.g., nitrogen deficiency or nitrogen limitation), nutrient excess, atmospheric pollution, herbicide, pesticide and UV irradiation.

Biotic stress is stress that occurs as a result of damage done to plants by other living organisms, such as bacteria, viruses, fungi, parasites, beneficial and harmful insects, weeds, and cultivated or native plants.

As used herein “pollen” refers to viable pollen that is able to fertilize the weed species of interest and therefore competes with native pollination.

According to some embodiments of the invention, the pollen is selected such that it is effective enough to compete with pollen of the target weed. According to some embodiments, the term “effective enough” refers to the elicitation of a seed set that is about similar in quantity to that obtained when using irradiated pollen or non-irradiated (native) of the same species as the target weed +/−0.1-5 fold (e.g., 0.4-5 fold). Alternatively, when native pollen competition does not exist, or very low levels of native pollen are present, pollination by the pollen used inhibits apomixis of weeds and by this reduces their quantities as well [Ribeiro et al. 2012].

According to a specific embodiment, the pollen exhibits susceptibility to a single growth condition e.g., herbicide, temperature.

According to a specific embodiment, the pollen exhibits susceptibility to multiple growth conditions e.g., different herbicides.

According to a specific embodiment, the pollen is non-genetically modified.

According to a specific embodiment, the pollen is genetically modified.

According to a specific embodiment, the pollen is of the same genus as the weed species of interest.

According to a specific embodiment, the pollen and the weed species of interest are of different genera.

For example the Amaranthaceae genus comprises several species redroot pigweed (Amaranthus retroflexus), smooth pigweed (Amaranthus hybridus), Powell pigweed (Amaranthus powellii), spiny pigweed (Amaranthus spinosus), tumble pigweed (Amaranthus albus), prostrate pigweed (Amaranthus blitoides), common waterhemp (Amaranthus rudis), tall waterhemp (Amaranthus tuberculatus), Palmer amaranth (Amaranthus palmeri), Amaranthus tuberculatus, Amaranthus thunbergii, Amaranthus graecizans or Amaranthus viridis.

According to a specific embodiment, the pollen is not of Amaranthus spinosus.

According to a specific embodiment, the pollen is not of Amaranthus spinosus when the female is Amaranthus palmeri.

An example for pollen, which is not of the same species as the target weed can be in the Amaranthus genus: A. retroflexus female X Irradiated A. palmeri pollen.

Examples for pollination within different genera can be for the Amaranthaceae family: B. scoparia female X Irradiated A. palmeri pollen, or C. album X Irradiated A. palmeri pollen.

Other examples are provided in Table 1 below.

TABLE 1
a non-exhaustive list of pollination pairs. Each pair
(a row in the table) is considered an embodiment.

| # | Female | Pollen | Classification |
|---|---|---|---|
| 1 | A. tuberculatus | Irradiated A. palmeri | Amaranthus Genus |
| 2 | A. retroflexus | Irradiated A. palmeri | Amaranthus Genus |
| 3 | A. albus | Irradiated A. palmeri | Amaranthus Genus |
| 4 | A. blitoides | Irradiated A. palmeri | Amaranthus Genus |
| 5 | A. hybridus | Irradiated A. palmeri | Amaranthus Genus |
| 6 | A. powellii | Irradiated A. palmeri | Amaranthus Genus |
| 7 | A. rudis | Irradiated A. palmeri | Amaranthus Genus |
| 9 | A. Palmeri | Irradiated A. retroflexus | Amaranthus Genus |
| 10 | A. tuberculatus | Irradiated A. retroflexus | Amaranthus Genus |
| 11 | A. albus | Irradiated A. retroflexus | Amaranthus Genus |
| 12 | A. blitoides | Irradiated A. retroflexus | Amaranthus Genus |
| 13 | A. hybridus | Irradiated A. retroflexus | Amaranthus Genus |
| 14 | A. powellii | Irradiated A. retroflexus | Amaranthus Genus |
| 15 | A. rudis | Irradiated A. retroflexus | Amaranthus Genus |
| 16 | A. spinosus | Irradiated A. retroflexus | Amaranthus Genus |
| 17 | A. viridis | Irradiated A. retroflexus | Amaranthus Genus |
| 18 | A. Palmeri | Irradiated A. tuberculatus | Amaranthus Genus |
| 19 | A. retroflexus | Irradiated A. tuberculatus | Amaranthus Genus |
| 20 | A. albus | Irradiated A. tuberculatus | Amaranthus Genus |
| 21 | A. blitoides | Irradiated A. tuberculatus | Amaranthus Genus |
| 22 | A. hybridus | Irradiated A. tuberculatus | Amaranthus Genus |
| 23 | A. powellii | Irradiated A. tuberculatus | Amaranthus Genus |
| 24 | A. rudis | Irradiated A. tuberculatus | Amaranthus Genus |
| 25 | A. spinosus | Irradiated A. tuberculatus | Amaranthus Genus |
| 26 | A. viridis | Irradiated A. tuberculatus | Amaranthus Genus |
| 27 | B. scoparia | Irradiated A. palmeri | Amaranthaceae Family |
| 28 | C. album | Irradiated A. palmeri | Amaranthaceae Family |
| 29 | B. scoparia | Irradiated C. album | Amaranthaceae Family |
| 30 | C. album | Irradiated B. scoparia | Amaranthaceae Family |
| 31 | B. scoparia | Irradiated A. tuberculatus | Amaranthaceae Family |
| 32 | C. album | Irradiated A. tuberculatus | Amaranthaceae Family |
| 33 | L. multiflorum | Irradiated L. rigidum | Lolium genus |
| 34 | L. perenne | Irradiated L. rigidum | Lolium genus |
| 35 | L. temulentum | Irradiated L. rigidum | Lolium genus |
| 36 | L. canariense | Irradiated L. rigidum | Lolium genus |
| 37 | L. persicum | Irradiated L. rigidum | Lolium genus |
| 38 | L. rigidum | Irradiated L. perenne | Lolium genus |
| 39 | A. pratensis | Irradiated | Alopecurus genus |
| | | A. myosuroides | |
| 40 | A. japonicus | Irradiated | Alopecurus genus |
| | | A. myosuroides | |
| 41 | A. aequalis | Irradiated | Alopecurus genus |
| | | A. myosuroides | |
| 42 | A. myosuroides | Irradiated A. japonicus | Alopecurus genus |
| 43 | L. rigidum | Irradiated A. myosuroides | Poaceae Family |
| 44 | A. myosuroides | Irradiated L. rigidum | Poaceae Family |
| 45 | A. confertiflora | Irradiated A. | Ambrosia genus |
| | | artemisiifolia | |
| 46 | A. psilostachya | Irradiated A. | Ambrosia genus |
| | | artemisiifolia | |
| 47 | A. trifida | Irradiated A. | Ambrosia genus |
| | | artemisiifolia | |
| 48 | A. artemisiifolia | Irradiated A. trifida | Ambrosia genus |
| 49 | A. artemisiifolia | Irradiated A. contertiflora | Ambrosia genus |
| 50 | C. bonariensis | Irradiated C. canadensis | Erigeron genus |
| 51 | C. canadensis | Irradiated C. bonariensis | Erigeron genus |
| 52 | A. artemisiifolia | Irradiated C. canadensis | Asteraceae family |
| 53 | C. canadensis | Irradiated A. | Asteraceae family |
| | | artemisiifolia | |

According to some embodiments, the pollen is not A. hybridus when the target plant is A. tuberculatus.

According to some embodiments, the weed species of interest is selected such that the female flowers mature before male flowers, ensuring a high level of outcrossing. According to some embodiments, the weed species of interest is Bassia scoparia (Kochia).

The pollen used according to some embodiments of the invention is irradiated pollen that will reduce the fitness of the target weed as explained above.

According to a specific embodiment, there is provided a method of producing pollen that reduces fitness of at least one weed species of interest, the method comprising treating the weed species of interest (e.g., seeds, seedlings, tissue/cells) or pollen thereof with an agent that reduces fitness.

According to a specific embodiment, the agent is radiation.

According to a specific embodiment, the method comprises harvesting pollen from the weed species of interest following treating with the agent that reduces the fitness.

According to other specific embodiments, the pollen may be first harvested and then treated with the agent (i.e., radiation) that reduces the fitness of the weed species of interest.

According to some embodiments the pollen used is mature pollen, e.g., at least 20%, 50%, or 80% of the pollen is mature.

According to some embodiments, mature pollen is collected following the natural process of anther dehiscence.

According to a specific embodiment the radiation is selected from the group consisting of X-ray radiation, gamma radiation, particle irradiation such as alpha, beta or other accelerated particle, UV radiation. The skilled artisan will know which agent to select.

According to a specific embodiment, the radiation is X-ray radiation.

According to a specific embodiment, the dose of radiation (X-ray) is 50-600 Gy, e.g., 100-400 Gy, 150-400 Gy, 100-300 Gy, 150-250 Gy, 100-250 Gy 150-300 Gy, e.g., 150, 200, 250 or 300 Gy, such as in the case of Amaranthus genus (e.g., A. palmeri).

According to a specific embodiment, growing the weed producing pollen that reduces fitness is effected in a large scale setting (e.g., hundreds to thousands m², 0.01-100 Acres).

According to some embodiments of the invention, the weed producing pollen comprises only male plants.

Harvesting pollen is well known in the art. For example, by the use of paper bags. Another example is taught in US20060053686, which is hereby incorporated by reference in its entirety.

Once pollen is obtained it can be stored for future use. Examples of storage conditions include, but are not; limited to, storage temperatures in Celsius degrees e.g., −196, −160, −130, −80, −20, −5, 0, 4, 20, 25, 30 or 35; percent of relative humidity e.g., 0, 10, 20, 30, 40, 50, 60, 70, 80, 90 or 100. Control over humidity can be achieved by using a dehydrating agent or a humidity chamber as known in the art. Additionally, the pollen can be stored in light or dark.

Alternatively, the pollen product of the present teachings is subjected to a post-harvest treatment.

Thus, according to an aspect of the invention there is provided a method of producing pollen for use in artificial pollination, the method comprising:
(a) obtaining pollen that reduces fitness of at least one weed species of interest, e.g., as described herein; and
(b) treating the pollen for use in artificial pollination.

Accordingly, there is provided a composition of matter comprising weed pollen that reduces fitness of at least one weed species of interest, the pollen having been treated for improving its use in artificial pollination.

Examples of such treatments include, but are not limited to coating, priming, formulating, chemical inducers, physical inducers [e.g., potential inducers include, but are not limited to, ethanol, hormones, steroids, (e.g., dexamethasone, glucocorticoid, estrogen, estradiol), salicylic acid, pesticides and metals such as copper, antibiotics such as but not limited to tetracycline, Ecdysone, ACEI, Benzothiadiazole and Safener, Tebufenozide or Methoxyfenozide], solvent solubilization, drying, heating, cooling and irradiating (e.g., gamma, UV, X-ray, particle).

Additional ingredients and additives can be advantageously added to the pollen composition of the present invention and may further contain sugar, potassium, calcium, boron, and nitrates. These additives may promote pollen tube growth after pollen distribution on flowering plants.

In some embodiments, the pollen composition of the present invention contains dehydrated or partially dehydrated pollen.

Thus, the pollen composition may comprise a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, a timed-release coating, a water-resistant coating, an antibiotic, a fungicide, a nematicide, a herbicide or a pesticide.

As used herein the term “about” refers to +10%.

The terms “comprises”, “comprising”, “includes”, “including”, “having” and their conjugates mean “including but not limited to”.

The term “consisting of”′ means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

Amaranthus genus: A. tuberculatus female X Irradiated A. palmeri pollen Experiment 98

### Materials and Method

### Plants Cultivation

The experiment was conducted during May-June in an indoor growing room in Rehovot, Israel. A. tuberculatus seeds were sown in germinating trays. Three weeks after the seedlings had emerged, they were transplanted into 5 L pots with a potting soil mixture. When plants began flowering, they were closely monitored daily to identify their sex at an early stage. Immediately after sex identification the females and males were separated and placed in different locations: male plants were grown in a net house and female plants were grown in an indoor growing room (conditions of 35°/25° C., photoperiod 16/8 day/night) where the pollination experiment was conducted. Upon maturation 6 A. tuberculatus female plants were selected for the experiment. A. palmeri seeds were sown and grown using the same protocol. Immediately after sex identification, the males were separated and placed in the net house for pollen production.

### Pollen Collection and Irradiation

Once the male plants of A. tuberculatus and A. palmeri started to flower, the pollen had been collected separately with a vacuum cleaner. A. Palmeri pollen was placed in a 15 cm petri dishes. The A. palmeri petri dish was irradiated by X-ray with a dose of 300 Gy. A. tuberculatus pollen was not irradiated.

### Pollination

To test the pollination potential of the irradiated A. palmeri pollen on A. tuberculatus, each of the six A. tuberculatus female plants were artificially pollinated with the irradiated A. palmeri pollen, while A. tuberculatus pollen served as a positive control. On each plant, one paper tube with 10 mg of irradiated A. palmeri pollen (later referred as “Cross with Palmer” treatment) and another paper tube with 10 mg of A. tuberculatus pollen (later referred as the “Control”) were carefully placed on an inflorescence, leaving it for 20 minutes before removing. In addition, 2 empty paper tubes in total with no pollen inside were placed on an additional spike before pollination (“blank”, to evaluate the level of natural pollination caused by pollen in the air) and another spike at the end of pollination process (“blank end”, pollination to evaluate the level of pollination caused unintentionally to non-target spikes, during the artificial pollination) in order to evaluate the pollen contamination level. Sixteen days after pollination, all the treated spikes were cut and dried. Seeds were harvested manually. Total seed counts per spike were measured, average weight of a single seed was calculated, and seed morphology was examined.

### Results

### Seed Set

As can be seen from Table 2, below, pollination of A. tuberculatus female spikes with irradiated A. palmeri pollen yielded at least the same amount of seeds as the pollination with A. tuberculatus pollen and even more.

To evaluate the quality of the seeds that were obtained, the average weight of a seed obtained from the cross of A. tuberculatus X irradiated A. palmeri (“Cross with palmer”) was calculated and compared to average weight of a seed obtaining from a cross of A. tuberculatus X A. tuberculatus (“Control”). Results demonstrated that the average weight of a seed obtained from the cross with irradiated palmer is almost 6 times lower than the control average seed weight indicating that the obtained seeds from the cross with palmeri treatment are probably aborted seeds as expected (Table 3).

TABLE 2
The average number of seeds
The values are of the average numbers of seeds
from 6 spikes, each one from a different female.

| Total | | | | Cross |
|---|---|---|---|---|
| number of | | Blank | | with |
| seeds | Blank | end | Control | Palmer |
| Average | 13.17 | 22.33 | 396.17 | 655.17 |
| STE | 6.50 | 10.07 | 110.46 | 151.85 |

TABLE 3
Average seed weight

| Avg Seed | | cross |
|---|---|---|
| Weight (mg) | Control | with Palmer |
| Average | 0.353 | 0.06 |
| STE | 0.025 | 0.005 |
| P-value | | 1.97E−07 |

### Germination Assay

In general, the seeds obtained from the cross with palmer with the irradiated pollen looked thin, partly empty and their color was light brown while the ones obtained from the control pollen looked more filled and had a darker brown/black color. A germination assay was conducted in order to estimate the different germination levels between the seeds obtained by artificial pollination with the A. palmeri irradiated pollen versus the ones obtained from artificial pollination with A. tuberculatus regular pollen. Seeds were separated according to their visual appearance into two subgroups from each category. In the control there were normal seeds versus seeds that appeared aborted whereas in the cross pollination with A. Palmeri pollen there were seeds that were suspected as “normal” versus seeds that appeared aborted. Up to 50 seeds from each group (according to the amount that was detected in each group) were taken from each of the samples, each set of seeds was sown in small pots for the germination test. These pots were placed in a growth chamber in 34/25° C. 16/8 h day/night conditions for 14 days. After 14 days, seedlings were counted, and germination rate was calculated for each sample. None of the seeds that were classified as aborted in both groups germinated. Regarding seeds that were classified as “normal”: while the average germination rate obtained from the control pollen was 44%, none of the seeds obtained from the. palmeri irradiated pollen germinated (Table 4).

TABLE 4
Germination assay

| | control | Cross with palmer |
|---|---|---|
| % Germination | normal | suspected as “normal” |
| Average | 0.44 | 0 |
| STE | 0.12 | 0 |
| P-value (control | | 0.0025 |
| compared to cross) | | |

### Example 2

Interspecific Pollen Application: A. Tuberculatus X A. Palmeri with Competition Experiment 720

### Experiment Aims

1. To test the pollination potential of irradiated A. palmeri pollen on A. tuberculatus when in competition with non-irradiated A. tuberculatus pollen.
2. To compare the pollination potential of irradiated A. palmeri pollen on A. tuberculatus to that of irradiated A. tuberculatus pollen, whether alone or in competition with non-irradiated A. tuberculatus pollen.

### Materials and Method

### Plant Cultivation

The experiment was conducted during August in Rehovot, Israel. A. tuberculatus seeds were sown in germination trays and grown within indoor growth chambers. Three weeks after the seedlings had emerged, they were transplanted into 5 L pots with a potting soil mixture. When plants began flowering, they were closely monitored daily to identify their sex at an early stage. Immediately after sex identification the females and males were separated and placed at different locations: male plants were grown in a net house and female plants were grown outdoors, approximately 300 meters from the male net house, where the pollination experiment was conducted. Upon maturation 3 A. tuberculatus female plants were selected for the experiment. A. palmeri seeds were sown and grown using the same protocol. Immediately after sex identification the males were separated and placed in a separate net house for pollen production.

### Pollen Collection and Irradiation

Once the male plants of A. tuberculatus and A. palmeri started to flower, pollen was collected separately for each species with a vacuum cleaner. A. tuberculatus pollen was divided into two batches originating from the same pollen mixture: one to be irradiated and the other to remain non-irradiated. Irradiation of A. tuberculatus and A. palmer pollen was carried out by placing the pollen in a 14 cm petri dish (A. palmer) or in a 1.5 mL Eppendorf tube (A. tuberculatus; because the amount was smaller than A. palmer pollen) and irradiating by X-ray at a dose of 250 Gy.

### Pollination

Each of the three A. tuberculatus female plants were pollinated with the following pollen treatments. Two inflorescences (‘spikes’) were pollinated for each treatment within the same plant.
1. Non-irradiated A. tuberculatus
2. Irradiated A. palmeri
3. Irradiated A. tuberculatus
4. Irradiated A. palmeri & non-irradiated A. tuberculatus (ratio of 1:1)
5. Irradiated A. tuberculatus & non-irradiated A. tuberculatus (ratio of 1:1)

Pollinations were carried out by covering each single spike with a 14 cm long paper tube containing 10 mg of pollen. Pollinations in which competition was tested between irradiated and non-irradiated pollen contained 10 mg of pollen from a pre-mixed stock containing A. tuberculatus pollen and either irradiated A. palmeri pollen or irradiated A. tuberculatus pollen at a ratio of 1:1. In addition, two empty paper tubes in total with no pollen inside were placed on an additional spike before pollination (“blank”) and on another inflorescence at the end of the pollination process (“blank end”) in order to evaluate the pollen contamination level.

Sixteen days after pollination, all the treated spikes were cut and dried. Seeds were harvested manually, total seed counts per spike were measured, and the average weight of a single seed was calculated.

### Results

### Seed Set

The total amount of seeds (both normal and defected) did not differ significantly between pollination treatments (ANOVA, p=0.12; Table 1), indicating that the pollination potential of irradiated pollen, whether A. palmeri or A. tuberculatus, does not fall from that of non-irradiated A. tuberculatus pollen.

### Seed Weight

Average seed weight differed significantly between treatments (ANOVA, p=0.001; Table 1), with seeds from irradiated A. palmeri pollen weighing the least (0.05 mg) and seeds from non-irradiated A. tuberculatus pollen weighing the most (0.19 mg). As seed weight is an indicator of seed development, the low seed weight of seeds from irradiated pollen suggests that these seeds were aborted. Average seed weight for irradiated pollen in competition with non-irradiated A. tuberculatus pollen was similar whether the irradiated pollen was A. palmeri or A. tuberculatus pollen (average seed weight of 0.15 and 0.16 mg, respectively). These results, which are statistically significant, indicate that the ability of irradiated A. palmeri pollen to pollinate A. tuberculatus female flowers does not fall from that of irradiated A. tuberculatus pollen when in competition with non-irradiated pollen.

TABLE 5
Total seed count (normal and aborted) and average seed weight,
for inflorescences (“spikes”) pollinated with non-irradiated
A. tuberculatus pollen, irradiated A. tuberculatus and A. palmeri
pollen and mixed irradiated and non-irradiated pollen.

| | | Average weight |
|---|---|---|
| | Average* total | of single seed |
| Treatment | seed count ± SEM | (mg) ± SEM |
| Blank | 120 ± 33 | 0.08 ± 0.01 |
| Blank end | 80 ± 30 | 0.08 ± 0.02 |
| Non-irradiated A. tuberculatus | 1180 ± 207 | 0.19 ± 0.01 |
| Irradiated A. palmeri | 617 ± 128 | 0.05 ± 0.00 |
| Irradiated A. tuberculatus | 935 ± 236 | 0.09 ± 0.04 |
| Irradiated A. palmeri & | 1003 ± 243 | 0.15 ± 0.03 |
| non-irradiated A. tuberculatus | | |
| (ratio of 1:1) | | |
| Irradiated A. tuberculatus & | 1206 ± 267 | 0.16 ± 0.01 |
| non-irradiated A. tuberculatus | | |
| (ratio of 1:1) | | |

*Values are averaged across six spikes (2 spikes per each of three plants).

### Example 3

### Amaranthus Genus: A. Retroflexus Female X Irradiated A. Palmeri Pollen

Done as in Example 1 or Example 2 only with the indicated pollen and target weed.

### Example 4

### Amaranthaceae Family: B. Scoparia Female X Irradiated A. Palmeri Pollen

Done as in Example 1 or Example 2 only with the indicated pollen and target weed.

### Example 5

Amaranthaceae Family: Chenopodium. Album Female X Irradiated A. Palmeri Pollen Experiment 683

### Experiment Aim

The aim of this experiment was to test the pollination potential of the irradiated A. palmeri pollen on Chenopodium album, a monoecious and self-compatible plant in the Amaranthaceae family but not within the Amaranthus genues.

### Materials and Method

### Plant Cultivation

The experiment was conducted during June at the Rehovot agricultural farm, Israel. C. album seeds were sown in germination trays and grown within indoor growth chambers. Three weeks after the seedlings had emerged, they were transplanted into 5 L pots with a potting soil mixture and grown in a net house until used for the experiment, a week later.

A. palmeri seeds were sown and grown using the same protocol. When plants began flowering, they were closely monitored daily to identify their sex at an early stage. Immediately after sex identification the males were separated and placed in a separate net house for pollen production.

### Pollen Collection and Irradiation

Once the male plants of A. palmeri started to flower, pollen was collected with a vacuum cleaner. Irradiation of A. palmeri pollen was carried out by placing the pollen in a 14 cm petri dish and irradiating by X-ray with a dose of 250 Gy.

### Pollination

Ten C. album plants were chosen in total, out of which five were artificially pollinated with irradiated A. palmeri pollen (treatment) and five were left without treatment (control). Each of the five treated plants was artificially pollinated with irradiated A. palmeri pollen three times, four days apart. The artificial pollination was carried out by “spraying” a mixture of irradiated pollen and talcum powder (at a ratio of 2:1), using a Mini Duster (©Kiwi Pollen), directly on all inflorescences. The five control C. album plants were not pollinated artificially, but instead left to self-pollinate. Treatment and control plants were placed in two separate areas of a single net house, in the agriculture farm in Rehovot.

As a positive control for the quality of the irradiated A. palmeri pollen, two inflorescences of a single A. palmeri female were artificially pollinated with the irradiated pollen as described above. Both inflorescences produced many aborted seeds (460 and 758), confirming the quality of the irradiated pollen.

### Seed Harvest and Germination

Inflorescences from each plant were harvested 14 days after the last pollination. Seeds were harvested manually and separated according to appearance to ‘normal’ seeds (dark brown and round) and ‘aborted’ seeds (yellow-light brown and flat).

To examine germination rate, the ‘normal’ seeds from each plant were sown in small soil pots and kept in a growth chamber (16 hours light, at 35° C. and 8 hours dark, at 25° C.). The seeds were kept refrigerated in water for a week prior to sowing to enhance germination. Germination was monitored throughout a period of 5 months.

### Preliminary Results

Germination rate of seeds that appeared ‘normal’ was significantly lower for plants that were artificially pollinated with irradiated A. palmeri pollen compared to control plants: 22.7±4.7% and 39.5±7.8%, respectively; one-sided t-test, p-value=0.05.

### Example 6

Lolium Genus: L. multiflorum Female X Irradiated L. Rigidum Pollen

Done as in Example 1 or Example 2 only with the indicated pollen and target weed.

### Example 7

Lolium Genus: L. perenne Female X Irradiated L. Rigidum Pollen

Done as in Example 1 or Example 2 only with the indicated pollen and target weed.

### Example 8

### Alopecurus Genus: A. Japonicus Female X Irradiated A. Myosuroides Pollen

Done as in Example 1 or Example 2 only with the indicated pollen and target weed.

### Example 9

### Poaceae Family: L. Rigidum Female X Irradiated A. Myosuroides Pollen

Done as in Example 1 or Example 2 only with the indicated pollen and target weed.

### Example 10

### Ambrosia Genus: A. Trifida Female X Irradiated A. Artemisiifolia Pollen

Done as in Example 1 or Example 2 only with the indicated pollen and target weed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of weed control, the method comprising artificially pollinating a weed species of interest with irradiated pollen that reduces fitness of said weed species of interest, said pollen being of a different species than that of said weed species of interest and capable of competing with pollen of said weed species of interest, wherein said artificially pollinating is effected under competition conditions with native pollen.

2. The method of claim 1, wherein said pollen consists of pollen of a different species than that of said weed species of interest.

3. The method of claim 1, wherein said artificially pollinating is effected in a large scale of at least 0.1 acre.

4. The method of claim 1, wherein said pollen is comprised in a formulation which allows pollen distribution by artificial pollination.

5. The method of claim 1, wherein said irradiated pollen is by y radiation.

6. The method of claim 1, wherein said irradiated pollen is by X radiation.

7. The method of claim 1, wherein said irradiated pollen is by alpha radiation.

8. The method of claim 1, wherein said pollen is of the same genus as said weed species of interest.

9. The method of claim 1, wherein at least one of said pollen and said weed species of interest is a hybrid.

10. The method of claim 1, wherein said pollen is non-genetically modified pollen.

11. The method of claim 1, wherein said pollen is genetically modified pollen.

12. The method of claim 1, wherein said pollen is of the Amaranthaceae family.

13. The method of claim 1, wherein said pollen is of the Amaranthus genus.

14. The method of claim 12, wherein said pollen and said weed species of interest are selected from the group consisting of A. palmeri and A. tuberculatus.

15. The method of claim 1, wherein said pollen is A. palmeri.

16. The method of claim 13, wherein said pollen is a hybrid of A. palmeri and A. tuberculatus.

17. The method of claim 13, wherein said weed species of interest is a hybrid of A. palmeri and A. tuberculatus.

18. The method of claim 1, wherein said pollen and said weed species of interest are selected from the group of pairs shown in Table 1.

19. The method of claim 1, wherein said capable of competing is by fertilizing said weed species of interest.
